(19) United States
(12) Patent Application Publication
(10) Pub. No.: US 2025/0028872 A1
(43) Pub. Date: Jan. 23, 2025

(54) INTELLIGENT MODELING METHOD OF SCR REACTION PROCESS OF DIESEL VEHICLE EXHAUST

(71) Applicant: CATARC Automotive Test Center (Tianjin) Co., Ltd, Tianjin (CN)

(72) Inventors: Zhenguo LI, Tianjin (CN); Maoxuan WANG, Tianjin (CN); Hanming WU, Tianjin (CN); Yuankai SHAO, Tianjin (CN); Xiaoning REN, Tianjin (CN); Kaixiang LI, Tianjin (CN); Zhengjun YANG, Tianjin (CN); Maodong FANG, Tianjin (CN); Lingwei MENG, Tianjin (CN); Wang ZHANG, Tianjin (CN); Qiang LIU, Tianjin (CN)

(73) Assignee: CATARC Automotive Test Center (Tianjin) Co., Ltd, Tianjin (CN)

(21) Appl. No.: 18/893,021

(22) Filed: Sep. 23, 2024

Related U.S. Application Data
(63) Continuation of application No. PCT/CN2023/134200, filed on Nov. 27, 2023.

(30) Foreign Application Priority Data
Feb. 1, 2023 (CN) 202310048902.1

(51) Int. Cl. G06F 30/15

(52) U.S. Cl. CPC G06F 30/15

(57) ABSTRACT

An intelligent modeling method of a Selective Catalytic Reduction (SCR) reaction process of diesel vehicle exhaust includes the following steps: S1: acquiring a time sequence data set of an SCR reaction process of diesel vehicle exhaust for modeling, where the time sequence data set includes an input variable sequence and an output variable sequence; S2: selecting a multi-layer type-II fuzzy neural network as an SCR reaction process model of diesel vehicle exhaust based on the input variable sequence and the output variable sequence obtained in Si. The intelligent modeling method of the SCR reaction process of diesel vehicle exhaust can effectively model dynamic characteristics of the SCR reaction process such as nonlinearity, time delay and uncertain interference, and improve the accuracy, robustness and generalization ability of the SCR reaction process model.

### CROSS-REFERENCE TO RELATED APPLICATIONS

This application is a continuation of International Application No. PCT/CN2023/134200, filed on Nov. 27, 2023, which claims priority to Chinese Patent Application No. 202310048902.1, filed on Feb. 1, 2023. The disclosures of the above-mentioned applications are hereby incorporated by reference in their entireties.

### TECHNICAL FIELD

The present disclosure belongs to the technical field of vehicle post-processing control, in particular to an intelligent modeling method of a Selective Catalytic Reduction (SCR) reaction process of diesel vehicle exhaust.

### BACKGROUND

An SCR system of diesel vehicle exhaust uses urea as a reducing agent, which reacts with NOₓ in exhaust under the action of a catalyst to generate harmless nitrogen (N₂) and water (H₂O), thus effectively reducing NOₓ emission from exhaust. The SCR reaction process is affected by the factors such as temperature, airspeed, gas composition, original engine exhaust, a urea injection amount and catalyst activity. The catalytic reduction reaction varies differently, which directly affects the effect of SCR emission reduction. Therefore, the accurate modeling of SCR reaction of diesel vehicle exhaust can help engineers understand the catalytic reduction reaction process in detail, which facilitates the design of an accurate and reasonable urea injection control strategy.

At present, the SCR reaction process of diesel vehicle exhaust is modeled based on a first principle, that is, a physical and chemical reaction mechanism. The disadvantage of such modeling method is that the physical and chemical reaction mechanism needs to calibrate parameters such as an activation energy coefficient, a gas constant and a gas molar mass for modelling. These parameters require a single-component chemical reaction calibration test under ideal test conditions. However, during the actual operation of diesel vehicles, many chemical reactions take place simultaneously at the exhaust tail, and the gas composition is complex, which is quite different from the reaction parameters calibrated under ideal conditions in the laboratory. At the same time, engineers need to have enough physical and chemical knowledge to use the physical and chemical reaction mechanism for modelling. All the main reactions and side reactions in the SCR reaction process of diesel vehicle exhaust can be accurately modeled, so that the established mechanism model cannot accurately reflect the SCR reaction process of diesel vehicle exhaust.

### SUMMARY

In view of this, the present disclosure aims to propose an intelligent modeling method of an SCR reaction process of diesel vehicle exhaust, which avoids using the physical and chemical reaction mechanism model for modeling and effectively models the SCR complex dynamic reaction process by modeling the input and output data of the SCR reaction process model.

In order to achieve the above purpose, the technical scheme of the present disclosure is realized as follows.

An intelligent modeling method of a Selective Catalytic Reduction (SCR) reaction process of diesel vehicle exhaust includes the following steps:
S1: acquiring a time sequence data set of an SCR reaction process of diesel vehicle exhaust for modeling, where the time sequence data set includes an input variable sequence and an output variable sequence;
S2: selecting a multi-layer type-II fuzzy neural network as an SCR reaction process model of diesel vehicle exhaust based on the input variable sequence and the output variable sequence obtained in S1;
stablishing the SCR reaction process model of diesel vehicle exhaust in the Step S2 includes the following steps:
A1: selecting a type-II fuzzy membership function parameter and a fuzzy subsequent layer membership function, selecting a descent algorithm, and determining the number of nodes of a multi-layer structure;
A2: identifying antecedent parameters of a type-II fuzzy neural network model in an SCR reaction interval of diesel vehicle exhaust using a gradient descent method;
A3: identifying antecedent parameters of the type-II fuzzy neural network model in the SCR reaction interval of diesel vehicle exhaust using a recursive least square method;
the input variable sequence in the Step S1 includes one or more of an NOₓ concentration at an SCR inlet, a urea injection amount, an NH₃ escape concentration after SCR and an SCR catalyst temperature;
the output variable sequence in the Step S1 includes an NOₓ concentration at an SCR outlet;
the time sequence of the input variable sequence and the output variable sequence in the Step S1 includes a sequence consisting of a measured value at a current moment and a measured value at a historical moment;
a method of acquiring the time sequence data in the Step S1 includes one of an engine bench test method and a vehicle field test method of a portable emission measurement system.

The multi-layer type-II fuzzy neural network model in the Step S2 includes an input layer, a type-II fuzzy layer, an interval operation layer, a fuzzy subsequent layer, a descent layer and an output layer.

The number of nodes in the input layer is one or two or more of time sequence data of an NOₓ concentration at an SCR inlet, a urea injection amount, an NH₃ escape concentration after SCR and an SCR catalyst temperature; the number of nodes of a type-II fuzzy layer, an interval operation layer and a fuzzy subsequent layer of the multi-layer type-II fuzzy neural network model in the Step S2 is the same, which is specified by artificial experience or obtained by parameter optimization.

The number of nodes of the descent layer of the multi-layer type-II fuzzy neural network model in the Step S2 is determined according to the descent algorithm; the descent algorithm includes a first descent algorithm and a second descent algorithm; the first descent algorithm determines that the number of nodes of the descent layer of the multi-layer type-II fuzzy neural network model is 2; the second descent algorithm determines that the number of nodes of the descent layer of the multi-layer type-II fuzzy neural network model is the same as that the number of nodes of the fuzzy subsequent layer.

Preferably, the first descent algorithm is an iterative algorithm, including any one of a Karnik-Mendel algorithm, an enhanced Karnik-Mendel algorithm, an enhanced Karnik-Mendel algorithm with new initialization algorithm, an iterative algorithm with stop condition algorithm, an enhanced iterative algorithm with stop condition algorithm, and an enhanced opposite direction searching algorithm.

Preferably, the second descent algorithm is a closed-loop algorithm, including any one of a Wu-Tan (WT) algorithm, an Nie-Tan (NT) algorithm, a Du-Ying (DY) algorithm and a Begian-Melek-Mendel (BMM) algorithm.

The type-II fuzzy membership function in the Step A1 includes one of an interval type-II Gaussian membership function, a generalized type-II Gaussian membership function, an interval type-II triangular membership function, a generalized type-II triangular membership function, an interval type-II trapezoidal membership function, a generalized type-II trapezoidal membership function, an interval type-II bell-shaped membership function and a generalized type-II bell-shaped membership function.

The fuzzy subsequent layer membership function of the type-II fuzzy neural network includes any one of a single-valued polynomial, an interval type-II fuzzy polynomial and a generalized type-II fuzzy polynomial.

Establishing the type-II fuzzy neural network model needs to calibrate the type-II fuzzy membership function parameter of the type-II fuzzy layer and the coefficients of the fuzzy subsequent layer polynomial.

A3 further includes using the time sequence data set of the SCR reaction process of diesel vehicle exhaust to train the type-II fuzzy neural network model antecedent parameter obtained in A2 and the type-II fuzzy neural network model subsequent parameter obtained in A3; and determining the parameters of the type-II fuzzy layer and the fuzzy subsequent layer of the type-II fuzzy neural network model calibrated to verify an minimum error and the descent algorithm as the multi-layer type-II fuzzy neural network model of the SCR reaction process of diesel vehicle exhaust.

A training algorithm includes one of a gradient descent method, a Newton method, a quasi-Newton method, a steepest descent method, a simulated annealing method, a genetic algorithm, an ant colony algorithm, a particle swarm algorithm, a least square method and a recursive least square method.

Compared with the prior art, the intelligent modeling method of the SCR reaction process of diesel vehicle exhaust has the following beneficial effects.

The intelligent modeling method of the SCR reaction process of diesel vehicle exhaust can avoid using the physical and chemical reaction mechanism for modeling and reduce model parameter calibration tests.

The intelligent modeling method of the SCR reaction process of diesel vehicle exhaust can effectively model dynamic characteristics of the SCR reaction process such as nonlinearity, time delay and uncertain interference, and improve the accuracy, robustness and generalization ability of the SCR reaction process model.

### BRIEF DESCRIPTION OF THE DRAWINGS

The accompanying drawings, which constitute a part of the present disclosure, are used to provide a further understanding of the present disclosure. The exemplary embodiments and the descriptions of the present disclosure are used to explain the present disclosure, rather than constitute an undue limitation of the present disclosure.

FIG. 1 is a schematic flow chart of an intelligent modeling method of an SCR reaction process of diesel vehicle exhaust according to Embodiment 1 of the present disclosure.

FIG. 2 is a schematic flow chart of a method of establishing an SCR reaction model of diesel vehicle exhaust according to the present disclosure.

FIG. 3 is a schematic flow chart of constructing an SCR reaction process model of diesel vehicle exhaust according to the present disclosure.

FIG. 4 is a first structure of a six-layer interval type-II fuzzy neural network model in Embodiment 1 of the present disclosure, which is an SCR reaction process model of diesel vehicle exhaust.

FIG. 5 is an interval type-II Gaussian fuzzy membership function of a six-layer interval type-II fuzzy neural network in Embodiment 1 of the present disclosure.

FIG. 6 is a second structure of a six-layer interval type-II fuzzy neural network model in Embodiment 2, which is an SCR reaction process model of diesel vehicle exhaust.

### DETAILED DESCRIPTION OF THE EMBODIMENTS

It should be noted that the embodiments in the present disclosure and the features in the embodiments can be combined with each other without conflict.

The present disclosure will be described in detail with reference to the accompanying drawings and embodiments.

### Embodiment 1

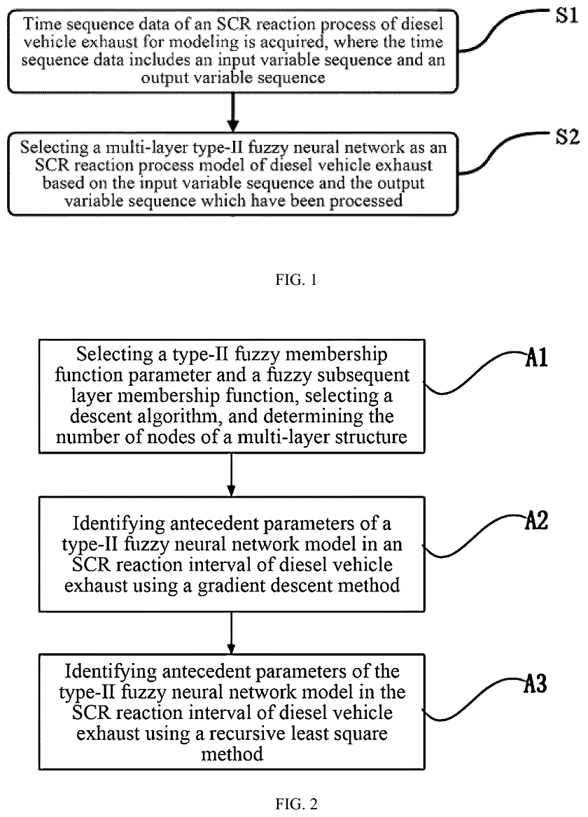

As shown in FIG. 1, the present disclosure provides an intelligent modeling method of a Selective Catalytic Reduction (SCR) reaction process of diesel vehicle exhaust, including:

S1: acquiring a time sequence data set of an SCR reaction process of diesel vehicle exhaust for modeling, wherein the time sequence data set includes an input variable sequence and an output variable sequence; and

S2: selecting a multi-layer type-II fuzzy neural network as an SCR reaction process model of diesel vehicle exhaust based on the input variable sequence and the output variable sequence obtained in S1.

As shown in FIG. 2, establishing the SCR reaction process model of diesel vehicle exhaust in the Step S2 includes the following steps:

A1: selecting a type-II fuzzy membership function parameter and a fuzzy subsequent layer membership function, selecting a descent algorithm, and determining the number of nodes of a multi-layer structure;

A2: identifying antecedent parameters of a type-II fuzzy neural network model in an SCR reaction interval of diesel vehicle exhaust using a gradient descent method; and

A3: identifying antecedent parameters of the type-II fuzzy neural network model in the SCR reaction interval of diesel vehicle exhaust using a recursive least square method.

The following examples are used to have a detailed description.

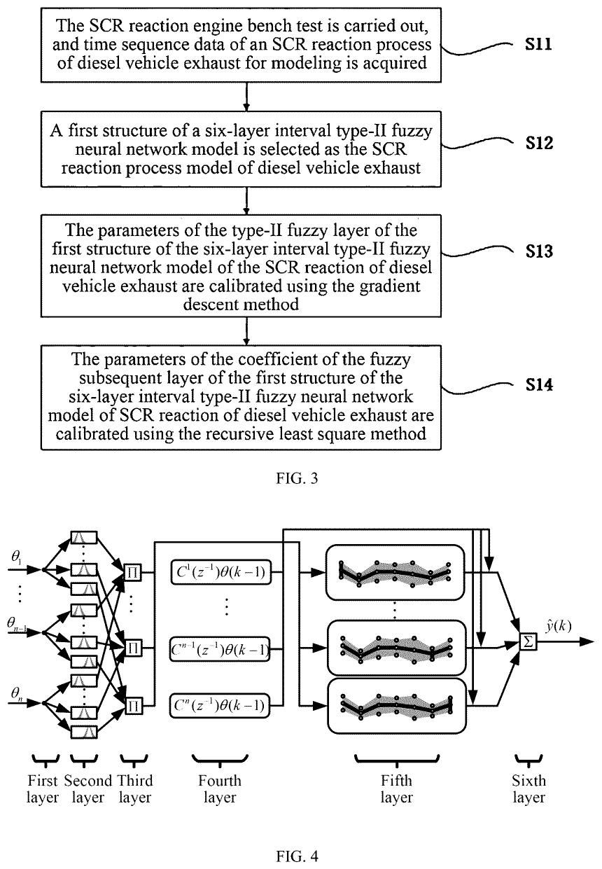

FIG. 1 is a schematic flow chart of an intelligent modeling method of an SCR reaction process of diesel vehicle exhaust according to Embodiment 1 of the present disclosure. The embodiment is suitable for constructing an SCR reaction process model of diesel vehicle exhaust. The method specifically includes the following steps as shown in FIG. 3.

S11: The SCR reaction engine bench test is carried out, and time sequence data of an SCR reaction process of diesel vehicle exhaust for modeling is acquired, where the time sequence data includes an input variable sequence and an output variable sequence.

Specifically, a diesel engine of 3 L in a main engine plant is used to carry out the test on the bench system of the AVL electric dynamometer of 330 kW. The direct emission sampling system is the AVL AMA i60 emission test system. The unconventional pollutants are measured by AVL FTIR. At the same time, an AVL 735/753 fuel consumption analyzer, a water constant temperature system and an intake air conditioning system are equipped. The input variable sequence and the output variable sequence are collected through the above devices.

Specifically, the input variable sequence includes a urea injection amount x₁, an NOₓ concentration x₂ at an SCR inlet, and an NH₃ escape concentration x₃ at the SCR outlet. The output variable sequence includes the NOₓ concentration y at the SCR outlet.

S12: A first structure of a six-layer interval type-II fuzzy neural network model in FIG. 4 is selected as the SCR reaction process model of diesel vehicle exhaust.

Specifically, the first layer of the first structure of the six-layer interval type-II fuzzy neural network model is the input layer, and the variables corresponding to n nodes are:

[θ₁, . . . ,θₙ]=[y(k−1), . . . ,y(k−n_{y}),x₁(k−1), . . . ,x₁(k−nₓ_{₁}),x₂(k−1), . . . x₂(k−nₓ_{₂})

x₃(k−1), . . . , x₃(k−nₓ_{₃})]. y(k−i) denotes the measured value of the NOₓ concentration at the SCR outlet at moment k−i, x₁(k−i) denotes the measured value of the urea injection amount at moment k−i, and x₂(k−i) denotes the measured value of the NOₓ concentration at the SCR inlet at moment k−i.

x₃(k−i) denotes the measured value of the NH₃ escape concentration after SCR at moment k−i. n_{y}, nₓ_{₁}, nₓ_{₂}, nₓ_{₃} denote the order of the NOₓ concentration y at the SCR outlet, the order of the urea injection amount x₁, the order of the NOₓ concentration x₂ at the SCR inlet, and the order of the NH₃ escape concentration x₃ at the SCR outlet. The method of identifying n_{y}, nₓ_{₁}, nₓ_{₂}, nₓ_{₃} includes but is not limited to the following methods: Akaike's Information Criterion (AIC), Bayesian Information Criterion (BIC) and Final Prediction Error (FPE).

Specifically, the second layer of the first structure of the six-layer interval type-II fuzzy neural network model is the type-II fuzzy layer, and the interval type-II Gaussian fuzzy membership function shown in FIG. 3 is selected. The interval type-II Gaussian fuzzy membership function corresponding to M×M nodes of the type-II fuzzy layer are respectively:

\tilde{F}₁¹, \tilde{F}₁², . . . , \tilde{F}₁^{M}, . . . , \tilde{F}ₙ¹, . . . , \tilde{F}ₙ^{M}. The output of the node of the type-II fuzzy layer is:

$$\{ \begin{matrix}{\overline{μ}}_{{{F}^{\text{?}}}_{\text{?}}^{\text{?}}} ( {θ}_{\text{?}} ) = {e}^{- \frac{{( {θ}_{\text{?}} - {m}_{\text{?}}^{\text{?}} )}^{2}}{2 {( {σ}_{\text{?}}^{\text{?}} + {δ}_{\text{?}}^{\text{?}} )}^{2}}} \\ {\underline{μ}}_{{{F}^{\text{?}}}_{\text{?}}^{\text{?}}} ( {θ}_{\text{?}} ) = {e}^{- \frac{{( {θ}_{\text{?}} - {m}_{\text{?}}^{\text{?}} )}^{2}}{2 {( {σ}_{\text{?}}^{\text{?}} - {δ}_{\text{?}}^{\text{?}} )}^{2}}}\end{matrix} \qquad ( 1 )$$

$$\text{?} \text{indicates text missing or illegible when filed}$$

where μ_{\tilde{F}}_{ᵢ}_{^{j}}(θᵢ) denotes an upper membership of the variable θᵢ in the interval type-II Gaussian fuzzy membership function \tilde{F}ᵢ^{j}; and μ_{\tilde{F}}_{ᵢ}_{^{j}}(θᵢ) denotes a lower membership of the variable θᵢ in the interval type-II Gaussian fuzzy membership function \tilde{F}ᵢ^{j}.

mᵢ^{j}, σᵢ^{j}, δᵢ^{j} denote a center value, an upper membership width and a width interval of the interval type-II Gaussian fuzzy membership function \tilde{F}ᵢ^{j}, respectively, which are adjustable parameters. Specifically, the third layer of the first structure of the six-layer interval type-II fuzzy neural network model is the interval operation layer. The output corresponding to j(j=1, . . . , k) nodes of the interval operation layer is:

$$\{ \begin{matrix}{\overline{f}}^{j} = {\overline{μ}}_{{{F}^{\text{?}}}_{1}^{j}} ( {θ}_{1} ) * {\overline{μ}}_{{{F}^{\text{?}}}_{2}^{j}} ( {θ}_{2} ) * … * {\overline{μ}}_{{{F}^{\text{?}}}_{n}^{j}} ( {θ}_{n} ) \\ {\underline{f}}^{j} = {\underline{μ}}_{{{F}^{\text{?}}}_{1}^{j}} ( {θ}_{1} ) * {\underline{μ}}_{{{F}^{\text{?}}}_{2}^{j}} ( {θ}_{2} ) * … * {\underline{μ}}_{{{F}^{\text{?}}}_{n}^{j}} ( {θ}_{n} )\end{matrix} \qquad ( 2 )$$

$$\text{?} \text{indicates text missing or illegible when filed}$$

where ι̅ⱼ denotes an upper membership interval operation value of the j-th node; ι^{j}; denotes the upper membership interval operation value of the j-th node; * denotes an operator, including but not limited to the following operations: multiplication ×; taking the minimum min, and taking maximum max; μ_{\tilde{F}}_{ᵢ}_{^{j}}(θᵢ) and μ_{\tilde{F}}_{ᵢ}_{^{j}}(θᵢ) denote the output of the node of the type-II fuzzy layer in Formula (1).

Specifically, the fourth layer of the first structure of the six-layer interval type-II fuzzy neural network model is the fuzzy subsequent layer. The fuzzy subsequent layer selects a single-valued polynomial, and the output corresponding to the j(j=1, . . . , k)-th node is:

$${C}^{j} ( {z}^{- 1} ) θ ( k - 1 ) \qquad ( 3 )$$

$$where$$

$$\{ \begin{matrix}{C}^{j} ( {z}^{- 1} ) = [ {A}^{j} ( {z}^{- 1} ) , {B}^{j} ( {z}^{- 1} ) , {D}_{1}^{j} ( {z}^{- 1} ) , {D}_{2}^{j} ( {z}^{- 1} ) ] \\ \begin{matrix}{A}^{j} ( {z}^{- 1} ) = {a}_{1}^{j} + {a}_{2}^{j} {z}^{- 1} + … + {a}_{{n}_{\text{?}}}^{j} {z}^{- ( {n}_{\text{?}} - 1 )} , \\ {B}^{j} ( {z}^{- 1} ) = {b}_{1}^{j} + {b}_{2}^{j} {z}^{- 1} + … + {b}_{{n}_{\text{?}}}^{j} {z}^{- ( {n}_{\text{?}} - 1 )}\end{matrix} \\ \begin{matrix}{D}_{1}^{j} ( {z}^{- 1} ) = {d}_{11}^{j} + {d}_{12}^{j} {z}^{- 1} + … + {d}_{1 {n}_{\text{?}}}^{j} {z}^{- ( {n}_{{\text{?}}_{2}} - 1 )} , \\ {D}_{2}^{j} ( {z}^{- 1} ) = {d}_{21}^{j} + {d}_{22}^{j} {z}^{- 1} + … + {d}_{2 {n}_{\text{?}}}^{j} {z}^{- ( {n}_{{\text{?}}_{2}} - 1 )}\end{matrix} \\ θ ( k - 1 ) = {[ y ( k - 1 ) , {x}_{1} ( k - 1 ) , {x}_{2} ( k - 1 ) , {x}_{3} ( k - 1 ) ]}^{T}\end{matrix}$$

$$\text{?} \text{indicates text missing or illegible when filed}$$

C^{j}(z⁻¹) denotes the polynomial coefficient vector of the j-th node; z⁻¹ is a discrete time sequence backward shift operator; and θ(k−1) is variable vector of the j-th node.

A^{j}(z⁻¹), B^{j}(z⁻¹), D₁^{j}(z⁻¹), D₂^{j}(z⁻¹) denote polynomial coefficients of the NOₓ concentration y at the SCR outlet, the urea injection amount x₁, the NOₓ concentration x₂ at the SCR inlet, and the NH₃ escape concentration x₃ at the SCR outlet, respectively;

$${a}_{1}^{j} , {a}_{2}^{j} , {a}_{{n}_{y}}^{j}$$

denote the first coefficient, the second coefficient and the n_{y}-th coefficient of the polynomial A^{j}(z⁻¹) of the NOₓ concentration y at the SCR outlet, respectively;

$${b}_{1}^{j} , {b}_{2}^{j} , {b}_{{n}_{{x}_{1}}}^{j}$$

denote the first coefficient, the second coefficient and the nₓ_{₁}-th coefficient of the polynomial of the urea injection amount x₁, respectively.

$${d}_{11}^{j} , {d}_{12}^{j} , {d}_{1 {n}_{{x}_{2}}}^{j}$$

denote the first coefficient, the second coefficient and the nₓ_{₂}-th coefficient of the polynomial D₁^{j}(z⁻¹) of the NOₓ concentration x₂ at the SCR inlet, respectively;

$${d}_{21}^{j} , {d}_{22}^{j} , {d}_{2 {n}_{{x}_{3}}}^{j}$$

denote the first coefficient, the second coefficient and the nₓ_{₃}-th coefficient of the polynomial D₂^{j}(z⁻¹) of the NH₃ escape concentration x₃ at the SCR outlet, respectively.

Specifically, the fifth layer of the first structure of the six-layer interval type-II fuzzy neural network model is the descent layer. The descent layer selects the NT descent algorithm, and the output corresponding to the j(j=1, . . . , k)-th node is:

$${ω}^{j} ( k ) [ {C}^{j} ( {z}^{- 1} ) θ ( k - 1 ) ] \qquad ( 4 )$$

where ωⱼ(k) denotes the normalized value of the membership of the j-th node at moment k, ω^{j}(k)=(ι̅^{j}+ι^{j})/Σⱼ₌₁^{M}(ι̅^{j}+ι^{j}), ι̅^{j} and ι^{j} denote the output of the node of the interval operation layer in Formula (2); Cⱼ(z⁻¹) and denote the output of the node of the fuzzy subsequent layer in Formula (3).

Specifically, the sixth layer of the first structure of the six-layer interval type-II fuzzy neural network model is the output layer. The output corresponding to the unique node of the output layer is:

$$\hat{y} ( k ) = {\sum }_{j = 1}^{M} {ω}^{j} ( k ) [ {C}^{j} ( {z}^{- 1} ) θ ( k - 1 ) ] + ξ ( k ) \qquad ( 5 )$$

where ŷ(k) denotes the predicted value of the NOₓ concentration at the SCR outlet at moment k; ξ(k) denotes the noise variable at moment k; ω^{j}(k)[C^{j}(z⁻¹)θ(k−1)] denotes the output of the node of the descent layer in Formula (4).

S13: The parameters of the type-II fuzzy layer of the first structure of the six-layer interval type-II fuzzy neural network model of the SCR reaction of diesel vehicle exhaust are calibrated using the gradient descent method.

Specifically, the error objective function of the first structure of the six-layer interval type-II fuzzy neural network model is selected as:

$${Θ}_{1} ( k ) = \frac{1}{2} {( y ( k ) - \hat{y} ( k ) )}^{2} = \frac{1}{2} {Δ}^{2} ( k ) \qquad ( 6 )$$

where Θ₁(k) denotes one half of the square of the error between the measured value of the NOₓ concentration at the SCR outlet and the predicted value of the NOₓ concentration at the SCR outlet at moment k; y(k) denotes the measured value of the NOₓ concentration at the SCR outlet at moment k; ŷ(k) denotes the predicted value of the NOₓ concentration at the SCR outlet at moment k; Δ(k)=y(k)−ŷ(k) denotes the predicted error of the NOₓ concentration at the SCR outlet at moment k.

Specifically, the parameter identification algorithm of the type-II fuzzy layer of the first structure of the six-layer interval type-II fuzzy neural network model is:

$${m}_{i}^{j} ( k + 1 ) = {m}_{i}^{j} ( k ) - η \frac{\partial {Θ}_{1} ( k )}{\partial {m}_{i}^{j} ( k )} = {m}_{i}^{j} ( k ) + η ( y ( k ) - \hat{y} ( k ) ) \frac{\partial \hat{y} ( k )}{\partial {m}_{i}^{j} ( k )} \qquad ( 7 )$$

$${σ}_{i}^{j} ( k + 1 ) = {σ}_{i}^{j} ( k ) - η \frac{\partial {Θ}_{1} ( k )}{\partial {σ}_{i}^{j} ( k )} = {σ}_{i}^{j} ( k ) + η ( y ( k ) - \hat{y} ( k ) ) \frac{\partial \hat{y} ( k )}{\partial {σ}_{i}^{j} ( k )}$$

$${δ}_{i}^{j} ( k + 1 ) = {δ}_{i}^{j} ( k ) - η \frac{\partial {Θ}_{1} ( k )}{\partial {δ}_{i}^{j} ( k )} = {δ}_{i}^{j} ( k ) + η ( y ( k ) - \hat{y} ( k ) ) \frac{\partial \hat{y} ( k )}{\partial {δ}_{i}^{j} ( k )}$$

$$\frac{\partial \hat{y} ( k )}{\partial {m}_{i}^{j} ( k )} = \frac{{ω}^{j} ( k ) [ {C}^{j} ( {z}^{- 1} ) θ ( k - 1 ) ] - \hat{y} ( k )}{{Σ}_{j = 1}^{L} ( {\overline{f}}^{j} + {\underline{f}}^{j} )} ( \frac{{\overline{f}}^{j} ( {θ}_{i} - {m}_{i}^{j} ( k ) )}{{( {σ}_{i}^{j} ( k ) + {δ}_{i}^{j} ( k ) )}^{2}} + \frac{{\underline{f}}^{j} ( {θ}_{i} - {m}_{i}^{j} ( k ) )}{{( {σ}_{i}^{j} ( k ) - {δ}_{i}^{j} ( k ) )}^{2}} )$$

$$\frac{\partial \hat{y} ( k )}{\partial {σ}_{i}^{j} ( k )} = \frac{{ω}^{j} ( k ) [ {C}^{j} ( {z}^{- 1} ) θ ( k - 1 ) ] - \hat{y} ( k )}{{Σ}_{j = 1}^{L} ( {\overline{f}}^{j} + {\underline{f}}^{j} )} ( \frac{{{\overline{f}}^{j} ( {θ}_{i} - {m}_{i}^{j} ( k ) )}^{2}}{{( {σ}_{i}^{j} ( k ) + {δ}_{i}^{j} ( k ) )}^{3}} + \frac{{{\underline{f}}^{j} ( {θ}_{i} - {m}_{i}^{j} ( k ) )}^{2}}{{( {σ}_{i}^{j} ( k ) - {δ}_{i}^{j} ( k ) )}^{3}} )$$

$$\frac{\partial \hat{y} ( k )}{\partial {δ}_{i}^{j} ( k )} = \frac{{ω}^{j} ( k ) [ {C}^{j} ( {z}^{- 1} ) θ ( k - 1 ) ] - \hat{y} ( k )}{{Σ}_{j = 1}^{L} ( {\overline{f}}^{j} + {\underline{f}}^{j} )} ( \frac{{{\overline{f}}^{j} ( {θ}_{i} - {m}_{i}^{j} ( k ) )}^{2}}{{( {σ}_{i}^{j} ( k ) + {δ}_{i}^{j} ( k ) )}^{3}} + \frac{{{\underline{f}}^{j} ( {θ}_{i} - {m}_{i}^{j} ( k ) )}^{2}}{{( {σ}_{i}^{j} ( k ) - {δ}_{i}^{j} ( k ) )}^{3}} )$$

where mᵢ^{j}(k+1) denotes the updated value of the center of the interval type-II Gaussian fuzzy membership function \tilde{F}ᵢ^{j} of the type-II fuzzy layer at moment k+1mᵢ^{j}(k) denotes the un-updated value of the center of the interval type-II Gaussian fuzzy membership function \tilde{F}ᵢ^{j} of the type-II fuzzy layer at moment k; σᵢ^{j}(k+1) denotes the updated value of the width of the interval type-II Gaussian fuzzy membership function \tilde{F}ᵢ^{j} of the type-II fuzzy layer at moment k+1; σᵢ^{j}(k) denotes the un-updated value of the width of the interval type-II Gaussian fuzzy membership function \tilde{F}ᵢ^{j} of the type-II fuzzy layer at moment k;

δᵢ^{j}(k+1) denotes the updated value of the width interval of the interval type-II Gaussian fuzzy membership function \tilde{F}ᵢ^{j}: of the type-II fuzzy layer at moment k+1; δᵢ^{j}(k) denotes the un-updated value of the width interval of the interval type-II Gaussian fuzzy membership function \tilde{F}ᵢ^{j}: of the type-II fuzzy layer at moment k; ∂Θ₁(k)/∂mᵢ^{j}(k) denotes the derivative of the error objective function Θ₁(k) on mᵢ^{j}(k) (in Formula (6); η denotes the learning rate of the gradient descent method, in which the value is in the range of 0 to 1; ŷ(k) denotes the predicted value of the NOₓ concentration at the SCR outlet at moment k, and ∂Θ₁(k)/∂mᵢ^{j}(k): denotes the derivative of the predicted value of the NOₓ concentration at the SCR outlet on mᵢ^{j}(k) at moment k.

S15: The parameters of the coefficient of the fuzzy subsequent layer of the first structure of the six-layer interval type-II fuzzy neural network model of SCR reaction of diesel vehicle exhaust are calibrated using the recursive least square method.

Specifically, the first structure of the six-layer interval type-II fuzzy neural network model of SCR reaction of diesel vehicle exhaust is rewritten in a vector form as:

$$\hat{y} ( k ) = {ξ}^{T} ( k - 1 ) \hat{β} ( k ) \qquad ( 8 )$$

$$ξ ( k - 1 ) = {[ {ξ}_{1}^{T} ( k - 1 ) , L , {ξ}_{M}^{T} ( k - 1 ) ]}^{T}$$

$$\hat{β} ( k ) = {[ {β}_{1}^{T} ( k ) , L , {β}_{M}^{T} ( k ) ]}^{T}$$

$${ξ}_{j} ( k - 1 ) = [ {ω}^{j} ( k ) y ( k - 1 ) , L , {ω}^{j} ( k ) y ( k - {n}_{y} ) , {ω}^{j} ( k ) {x}_{1} ( k - 1 ) , L , {ω}^{j} ( k ) {x}_{1} ( k - {n}_{{x}_{1}} ) ,$$

$${{ω}^{j} ( k ) {x}_{2} ( k - 1 ) , L , {ω}^{j} ( k ) {x}_{2} ( k - {n}_{{x}_{2}} ) , {ω}^{j} ( k ) {x}_{3} ( k - 1 ) , L , {ω}^{j} ( k ) {x}_{3} ( k - {n}_{{x}_{3}} ) ]}^{T}$$

$${β}_{j} ( k ) = {[ {a}_{1}^{j} , L , {a}_{{n}_{y}}^{j} {b}_{1}^{j} , L , {b}_{{n}_{x 1}}^{j} , {d}_{11}^{j} , L , {d}_{1 {n}_{x 2}}^{j} , {d}_{21}^{j} , L , {d}_{2 {n}_{x 3}}^{j} ]}^{T}$$

where ξ(k−1) denotes the known parameter vector at moment k−1; ξⱼ^{T}(k−1) denotes the j-th known parameter element at moment k−1, j=1, K, M; \hat{β}(k). denotes the coefficient vector of the fuzzy subsequent layer that needs to be updated at moment k; βⱼ(k), denotes the j-th coefficient element of the fuzzy subsequent layer that needs to be updated at moment k, j=1, K, M; ω^{j}(k) denotes the normalized value of the membership of the j-th node at moment k; y(k−i) denotes the measured value of the NOₓ concentration at the SCR outlet at moment k−i. x₁(k−i) denotes the measured value of urea injection amount at moment k−i; x(k−i) denotes the measured value of the NOₓ concentration at the SCR inlet at moment k−i; x₃(k−i) denotes the measured value of the NH₃ escape concentration after SCR at moment k−i;

$${a}_{1}^{j} , {a}_{2}^{j} , {a}_{{n}_{y}}^{j}$$

denote the first coefficient, the second coefficient and the n_{y}-th coefficient of the polynomial A^{j}(z⁻¹) of the NOₓ concentration y at the SCR outlet, respectively;

$${b}_{1}^{j} , {b}_{2}^{j} , {b}_{{n}_{{x}_{1}}}^{j}$$

denote the first coefficient, the second coefficient and the n_{z}_{₁}-th coefficient of the polynomial B^{j}(z⁻¹) of the urea injection amount x₁, respectively;

$${d}_{11}^{j} , {d}_{12}^{j} , {d}_{1 {n}_{{x}_{2}}}^{j}$$

denote the first coefficient, the second coefficient and the nₓ_{₂}-th coefficient of the polynomial Dᵢ^{j}(z⁻¹) of the NOₓ concentration x₂ at the SCR inlet, respectively;

$${d}_{21}^{j} , {d}_{22}^{j} , {d}_{2 {n}_{{x}_{3}}}^{j}$$

denote the first coefficient, the second coefficient and the nₓ_{₃}-th coefficient of the polynomial D₂^{j}(z⁻¹) of the NH₃ escape concentration x₃ at the SCR outlet, respectively.

The fuzzy subsequent layer coefficient vector \hat{β}(k) that needs to be updated is iteratively updated using the following recursive least square method:

$$\begin{matrix}\hat{β} ( k ) = \hat{β} ( k - 1 ) + K ( k ) [ y ( k ) - {ξ}^{T} ( k - 1 ) \hat{β} ( k - 1 ) ] \\ K ( k ) = \frac{P ( k - 1 ) ξ ( k - 1 )}{1 + {ξ}^{T} ( k - 1 P ( k - 1 ) ξ ( k - 1 )} \\ P ( k ) = P ( k - 1 ) - K ( k ) {ξ}^{T} ( k - 1 ) P ( k - 1 )\end{matrix} \qquad ( 9 )$$

where \hat{β}(k) denotes the coefficient vector of the fuzzy subsequent layer that needs to be updated at moment k; \hat{β}(k−1) denotes the coefficient vector of the fuzzy subsequent layer at moment k−1; K(k) denotes the known gain matrix at moment k; P(k−1) denotes the covariance matrix at moment k−1; P(k) denotes the covariance matrix updated at moment k.

Further, the steps of calibrating parameters of S13 and S14 are repeated until the error objective number in Formula (6) satisfies: Θ₁(k)≤λ₁(10).

where λ₁ denotes the prediction accuracy of the first structure of the six-layer interval type-II fuzzy neural network model.

Specifically, after the prediction accuracy of the first structure of the six-layer interval type-II fuzzy neural network model satisfies Formula (10), the parameters of the type-II fuzzy layer in the Formula (7) and the parameters of the fuzzy subsequent layer in the Formula (9) are fixed parameters of the first structure of the six-layer interval type-II fuzzy neural network model. In this way, the modeling of the SCR reaction process of diesel vehicle exhaust is completed.

### Embodiment 2

FIG. 1 is a schematic flow chart of an intelligent modeling method of an SCR reaction process of diesel vehicle exhaust according to Embodiment 1 of the present disclosure. The embodiment is suitable for constructing an SCR reaction process model of diesel vehicle exhaust. The method specifically includes the following steps as shown in FIG. 3.

S11: The SCR reaction engine bench test is carried out, and time sequence data of an SCR reaction process of diesel vehicle exhaust for modeling is acquired, where the time sequence data includes an input variable sequence and an output variable sequence.

Specifically, a diesel engine of 3 L in a main engine plant is used to carry out the test on the bench system of the AVL electric dynamometer of 330 kW. The direct emission sampling system is the AVL AMA i60 emission test system. The unconventional pollutants are measured by AVL FTIR. At the same time, an AVL 735/753 fuel consumption analyzer, a water constant temperature system and an intake air conditioning system are equipped. The input variable sequence and the output variable sequence are collected through the above devices.

Specifically, the input variable sequence includes a urea injection amount x₁, an NOₓ concentration x₂ at an SCR inlet, and an NH₃ escape concentration x₃ at the SCR outlet. The output variable sequence includes the NOₓ concentration y at the SCR outlet.

S12: A first structure of a six-layer interval type-II fuzzy neural network model in FIG. 6 is selected as the SCR reaction process model of diesel vehicle exhaust.

Specifically, the first layer of the first structure of the six-layer interval type-II fuzzy neural network model is the input layer, and the variables corresponding to n nodes are:

[ƒ₁, . . . θₙ]=[y(k−1), . . . ,y(k−n_{y}),x₁(k−1), . . . ,x₁(k−nₓ_{₁}),x₂(k−1), . . . ,x₂(k−nₓ_{₂})

x₃(k−1), . . . , x₃(k−nₓ_{₃})]. y(k−i) denotes the measured value of the NOₓ concentration at the SCR outlet at moment k−i, x₁(k−i) denotes the measured value of the urea injection amount at moment k−i, and x₂(k−i) denotes the measured value of the NOₓ concentration at the SCR inlet at moment k−i. x₃(k−i) denotes the measured value of the NH₃ escape concentration after SCR at moment k−i. n_{y}, nₓ_{₁}, nₓ_{₂}, nₓ_{₃} denote the order of the NOₓ concentration y at the SCR outlet, the order of the urea injection amount x₁, the order of the NOₓ concentration x₂ at the SCR inlet, and the order of the NH₃ escape concentration x₃ at the SCR outlet. The method of identifying n_{y}, nₓ_{₁}, nₓ_{₂}, nₓ_{₃} includes but is not limited to the following methods: Akaike's Information Criterion (AIC), Bayesian Information Criterion (BIC) and Final Prediction Error (FPE).

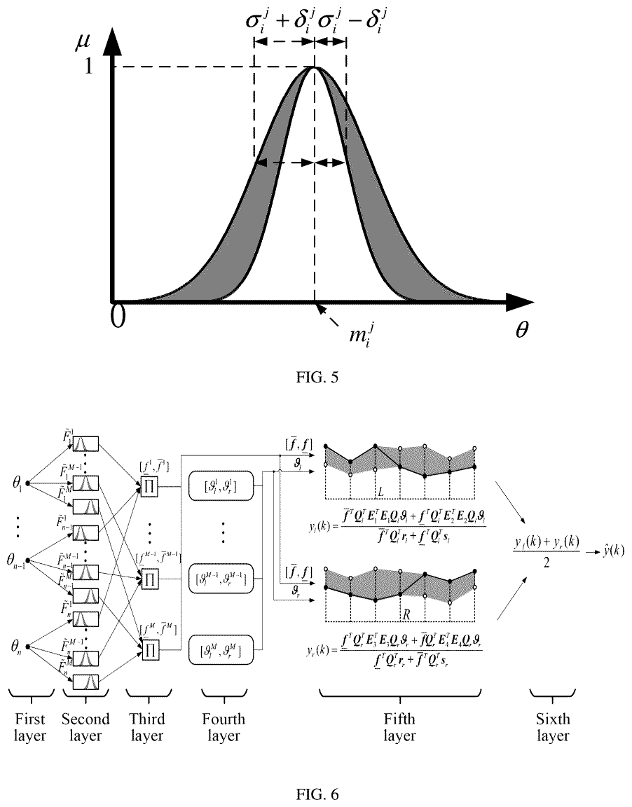

Specifically, the second layer of the first structure of the six-layer interval type-II fuzzy neural network model is the type-II fuzzy layer, and the interval type-II Gaussian fuzzy membership function shown in FIG. 5 is selected. The interval type-II Gaussian fuzzy membership function corresponding to n×M nodes of the type-II fuzzy layer are respectively:

\tilde{F}₁¹, \tilde{F}₁², . . . , \tilde{F}₁^{M}, . . . , \tilde{F}ₙ¹, . . . , \tilde{F}ₙ^{M}. The output of the node of the type-II fuzzy layer is:

$$\{ \begin{matrix}{\overline{μ}}_{{\tilde{F}}_{i}^{j}} ( {θ}_{i} ) = {e}^{- \frac{{( {θ}_{i} - {m}_{i}^{j} )}^{2}}{2 {( {σ}_{i}^{j} + {δ}_{i}^{j} )}^{2}}} \\ {\underline{μ}}_{{\tilde{F}}_{i}^{j}} ( {θ}_{i} ) = {e}^{- \frac{{( {θ}_{i} - {m}_{i}^{j} )}^{2}}{2 {( {σ}_{i}^{j} + {δ}_{i}^{j} )}^{2}}}\end{matrix} \qquad ( 11 )$$

where μ_{\hat{F}}_{ᵢ}_{^{j}}(θᵢ) denotes an upper membership of the variable θᵢ in the interval type-II Gaussian fuzzy membership function \tilde{F}ᵢ^{j}; μ_{F}_{ᵢ}_{^{j}}(θᵢ) denotes a lower membership of the variable θᵢ in the interval type-II Gaussian fuzzy membership function \tilde{F}ᵢ^{j}; mᵢ^{j}, σᵢ^{j}, δᵢ^{j} denote a center value, an upper membership width and a width interval of the interval type-II Gaussian fuzzy membership function \tilde{F}ᵢ^{j}, respectively, which are adjustable parameters.

Specifically, the third layer of the first structure of the six-layer interval type-II fuzzy neural network model is the interval operation layer. The output corresponding to j(j=1, . . . , M) nodes of the interval operation layer is:

$$\{ \begin{matrix}{\overline{f}}^{j} = {\overline{µ}}_{{\tilde{F}}_{1}^{j}} ( {θ}_{1} ) ⋆ {\overline{µ}}_{{\tilde{F}}_{2}^{j}} ( {θ}_{2} ) ⋆ … ⋆ {\overline{µ}}_{{\tilde{F}}_{n}^{j}} ( {θ}_{n} ) \\ {\underline{f}}^{j} {\underline{µ}}_{{\tilde{F}}_{1}^{j}} ( {θ}_{1} ) ⋆ {\underline{µ}}_{{\tilde{F}}_{2}^{j}} ( {θ}_{2} ) ⋆ … ⋆ {\underline{µ}}_{{\tilde{F}}_{n}^{j}} ( {θ}_{n} )\end{matrix} \qquad ( 12 )$$

where ι̅^{j} denotes an upper membership interval operation value of the j-th node; ι^{j}. denotes the upper membership interval operation value of the j-th node; * denotes an operator, including but not limited to the following operations: multiplication, taking the minimum min, and taking maximum max; μ̅_{\tilde{F}}_{ᵢ}_{^{j}}(θᵢ) and μ_{\tilde{F}}_{ᵢ}_{^{j}}(θᵢ) denote the output of the node of the type-II fuzzy layer in Formula (1).

Specifically, the fourth layer of the first structure of the six-layer interval type-II fuzzy neural network model is the fuzzy subsequent layer. The fuzzy subsequent layer selects a single-valued polynomial, and the output corresponding to the j(j=1, . . . , M)-th node is

$${C}^{j} ( {z}^{- 1} ) θ ( k - 1 ) = [ {C}_{l}^{j} ( {z}^{- 1} ) θ ( k - 1 ) , {C}_{r}^{j} ( {z}^{- 1} ) θ ( k - 1 ) ] \qquad ( 13 )$$

$$where$$

$$\{ \begin{matrix}{C}_{l}^{j} ( {z}^{- 1} ) = [ {A}_{l}^{j} ( {z}^{- 1} ) , {B}_{l}^{j} ( {z}^{- 1} ) , {D}_{l}^{j} ( {z}^{- 1} ) , {D}_{l}^{j} ( {z}^{- 1} ) ] \\ {C}_{r}^{j} ( {z}^{- 1} ) = [ {A}_{r}^{j} ( {z}^{- 1} ) , {B}_{r}^{j} ( {z}^{- 1} ) , {D}_{r}^{j} ( {z}^{- 1} ) , {D}_{r}^{j} ( {z}^{- 1} ) ] \\ {A}_{l}^{j} ( {z}^{- 1} ) = {a}_{1 , l}^{j} + {a}_{2 , l}^{j} {z}^{- 1} + ⋯ + {a}_{{n}_{y , l}}^{j} {z}^{- ( {n}_{y} - 1 )} , {A}_{r}^{j} ( {z}^{- 1} ) = {a}_{1 , r}^{j} + {a}_{2 , r}^{j} {z}^{- 1} + ⋯ + {a}_{{n}_{y , r}}^{j} {z}^{- ( {n}_{y} - 1 )} \\ {B}_{l}^{j} ( {z}^{- 1} ) = {b}_{1 , l}^{j} + {b}_{2 , l}^{j} {z}^{- 1} + ⋯ + {b}_{{n}_{{x}_{1} , l}}^{j} {z}^{- ( {n}_{{x}_{1}} - 1 )} {B}_{l}^{j} ( {z}^{- 1} ) = {b}_{1 , r}^{j} + {b}_{2 , r}^{j} {z}^{- 1} + ⋯ + {b}_{{n}_{{x}_{1} , r}}^{j} {z}^{- ( {n}_{{x}_{1}} - 1 )} \\ {D}_{1 , l}^{j} ( {z}^{- 1} ) = {d}_{11 , l}^{j} + {d}_{12 , l}^{j} {z}^{- 1} + ⋯ + {d}_{1 {n}_{x , 2} , l}^{j} {z}^{- ( {n}_{{x}_{2}} - 1 )} , {D}_{1 , r}^{j} ( {z}^{- 1} ) = {d}_{11 , r}^{j} + {d}_{12 , r}^{j} {z}^{- 1} + ⋯ + {d}_{1 {n}_{{x}_{2} , r}}^{j} {z}^{- ( {n}_{{x}_{2}} - 1 )} \\ {D}_{2 , l}^{j} ( {z}^{- 1} ) = {d}_{21 , l}^{j} + {d}_{22 , l}^{j} {z}^{- 1} + ⋯ + {d}_{2 {n}_{{x}_{3} , l}}^{j} {z}^{- ( {n}_{{x}_{3}} - 1 )} , {D}_{2 , r}^{j} ( {z}^{- 1} ) = {d}_{21 , r}^{j} + {d}_{22 , r}^{j} {z}^{- 1} + ⋯ + {d}_{2 {n}_{{x}_{3} , r}}^{j} {z}^{- ( {n}_{{x}_{3}} - 1 )} \\ θ ( k - 1 ) = {[ y ( k - 1 ) , {x}_{1} ( k - 1 ) , {x}_{2} ( k - 1 ) , {x}_{3} ( k - 1 ) ]}^{T}\end{matrix}$$

C^{j}(z⁻¹=[Cᵢ^{j}(z⁻¹), Cᵣ^{j}(z⁻¹)] denotes the polynomial coefficient vector of the j-th node, Cᵢ^{j}(z⁻¹) denotes the left interval polynomial coefficient vector of the j-th node, Cᵣ^{j}(z⁻¹) denotes the right interval polynomial coefficient vector of the j-th node; z⁻¹ is a discrete time sequence backward shift operator; ∝(k−1) is variable vector of the j-th node; Aᵢ^{j}(z⁻¹), Bᵢ^{j}(z⁻¹), D_{1,i}^{j}(z⁻¹), D_{2,i}^{j}(z⁻¹) denote the left interval polynomial coefficients of the NOₓ concentration y at the SCR outlet, the urea injection amount x₁, the NOₓ concentration x₂ at the SCR inlet, and the NH₃ escape concentration x₃ at the SCR outlet, respectively;

$${a}_{1 , l}^{j} , {a}_{2 , l}^{j} , {a}_{{n}_{x} , l}^{j}$$

denote the first coefficient, the second coefficient and the n_{y}-th coefficient of the left interval polynomial Aᵢ^{j}(z⁻¹) of the NOₓ concentration y at the SCR outlet, respectively;

$$a \text{?} , a \text{?} , a \text{?}$$

$$\text{?} \text{indicates text missing or illegible when filed}$$

denote the first coefficient, the second coefficient and the n_{y}-th coefficient of the right interval polynomial Aᵣ^{j}(z⁻¹) of the NOₓ concentration at the SCR outlet, respectively;

$$b \text{?} , b \text{?} , b \text{?}$$

$$\text{?} \text{indicates text missing or illegible when filed}$$

denote the first coefficient, the second coefficient and the nₓ_{₁}-th coefficient of the left interval polynomial Bᵢ^{j}(z⁻¹) of the urea injection amount x₁, respectively.

$$b \text{?} , b \text{?} , b \text{?}$$

$$\text{?} \text{indicates text missing or illegible when filed}$$

denote the first coefficient, the second coefficient and the nₓ_{₁}-th coefficient of the right interval polynomial Bᵢ^{j}(z⁻¹) of the urea injection amount x₁, respectively.

$$d \text{?} , d \text{?} , d \text{?}$$

$$\text{?} \text{indicates text missing or illegible when filed}$$

denote the first coefficient, the second coefficient and the nₓ_{₂}-th coefficient of the left interval polynomial D_{i,j}^{j}(z⁻¹) of the NOₓ concentration x₂ at the SCR outlet, respectively.

$$d \text{?} , d \text{?} , d \text{?}$$

$$\text{?} \text{indicates text missing or illegible when filed}$$

denote the first coefficient, the second coefficient and the nₓ_{₂}-th coefficient of the right interval polynomial D_{l,y}^{j}(z⁻¹) of the NOₓ concentration x₂ at the SCR inlet, respectively;

$$d \text{?} , d \text{?} , d \text{?}$$

$$\text{?} \text{indicates text missing or illegible when filed}$$

denote the first coefficient, the second coefficient and the nₓ_{₃}-th coefficient of the left interval polynomial D_{2,j}^{j}(z⁻¹) of the NH₃ escape concentration x₃ at the SCR outlet, respectively.

$$d \text{?} , d \text{?} , d \text{?}$$

$$\text{?} \text{indicates text missing or illegible when filed}$$

denote the first coefficient, the second coefficient and the nₓ_{₃}-th coefficient of the right interval polynomial D_{2,y}^{j}(z⁻¹) of the NH₃ escape concentration x₃ at the SCR outlet, respectively.

Specifically, the fifth layer of the second structure of the six-layer interval type-II fuzzy neural network model is the descent layer. The descent layer selects the KM descent algorithm, and the output corresponding to the left and right nodes of the descent layer are:

$$\{ \begin{matrix}{y}_{l} ( k ) = \frac{{\overline{f}}^{T} {Q}_{l}^{T} {E}_{1}^{T} {E}_{1} {Q}_{l} {ϑ}_{l} + {\underline{f}}^{T} {Q}_{l}^{T} {E}_{2}^{T} {E}_{2} {Q}_{l} {ϑ}_{l}}{{\overline{f}}^{T} {Q}_{l}^{T} {r}_{l} + {\underline{f}}^{T} {Q}_{l}^{T} {s}_{l}} \\ {y}_{r} ( k ) = \frac{{\underline{f}}^{T} {Q}_{r}^{T} {E}_{3}^{T} {E}_{3} {Q}_{r} {ϑ}_{r} + {\overline{f}}^{T} {Q}_{r}^{T} {E}_{4}^{T} {E}_{4} {Q}_{r} {ϑ}_{r}}{{\underline{f}}^{T} {Q}_{r}^{T} {r}_{l} + {\overline{f}}^{T} {Q}_{r}^{T} {s}_{l}}\end{matrix} \qquad ( 14 )$$

$$where$$

$$\{ \begin{matrix}{ϑ}_{l} = [ {ϑ}_{l}^{1} , {ϑ}_{l}^{2} , … , {ϑ}_{l}^{M} ] = [ {{C}_{l}}^{1} ( {z}^{- 1} ) θ ( k - 1 ) , {{C}_{l}}^{2} ( {z}^{- 1} ) θ ( k - 1 ) , … , {{C}_{l}}^{M} ( {z}^{- 1} ) θ ( k - 1 ) ] \\ {ϑ}_{r} = [ {ϑ}_{r}^{1} , {ϑ}_{r}^{2} , … , {ϑ}_{r}^{M} ] = [ {{C}_{r}}^{1} ( {z}^{- 1} ) θ ( k - 1 ) , {{C}_{r}}^{2} ( {z}^{- 1} ) θ ( k - 1 ) , … , {{C}_{r}}^{M} ( {z}^{- 1} ) θ ( k - 1 ) ] \\ \overline{f} = [ {\overline{f}}^{1} , {\overline{f}}^{2} , … , {\overline{f}}^{M} ] \\ \underline{f} = [ {\underline{f}}^{1} , {\underline{f}}^{2} , … , {\underline{f}}^{M} ] \\ {E}_{1} = [ {e}_{1 ,}^{1} {e}_{2}^{1} , … , {e}_{L}^{1} , 0 , … , 0 ] ϵ {ℝ}^{L \times M} , {E}_{2} = [ 0 , … , 0 , {ε}_{1 ,}^{2} {ε}_{2}^{2} , … , {ε}_{M - L}^{2} ] ϵ {ℝ}^{( M - L ) \times M} \\ {E}_{3} = [ {e}_{1 ,}^{3} {e}_{2}^{3} , … , {e}_{R}^{3} , 0 , … , 0 ] ϵ {ℝ}^{R \times M} , {E}_{4} = [ 0 , … , 0 , {ε}_{1 ,}^{4} {ε}_{1}^{4} , … , {ε}_{M - R}^{4} ] ϵ {ℝ}^{( M - R ) \times M} ] \\ {r}_{l} = {\underbrace{[ 1 , 1 , … 1 ,}}_{L} 0 , 0 , … , 0 {]}^{T} ϵ {ℤ}^{M} , {s}_{l} = [ 0 , 0 , … , 0 {{\underbrace{1 , 1 , … 1 , ]}}_{M - L}}^{T} ϵ {ℤ}^{M} \\ {r}_{r} = {\underbrace{[ 1 , 1 , … 1 ,}}_{R} 0 , 0 , … , 0 {]}^{T} ϵ {ℤ}^{M} , {s}_{r} = [ 0 , 0 , … , 0 {{\underbrace{1 , 1 , … 1 , ]}}_{M - R}}^{T} ϵ {ℤ}^{M}\end{matrix} ;$$

yᵢ(k) denotes the output of the node on the left side of the descent layer at moment k; yᵣ(k) denotes the output of the node on the right side of the descent layer at moment k; ϑₗ denotes the parameter vector on the left side of the fuzzy subsequent layer, ϑₗ^{j}=Cₗ^{j}(z⁻¹)θ(k−1) denotes the parameter output on the left side of the j-th fuzzy subsequent layer node in Formula (13); ϑᵣ denotes the parameter vector on the right side of the fuzzy subsequent layer, ϑₗ^{j}=Cₗ^{j}(z⁻¹)θ(k−1) denotes the parameter output on the right side of the j-th node of the fuzzy subsequent layer in Formula (13); ι̅ denotes the upper interval output vector of the node of the interval operation layer in Formula (12); ι denotes the lower interval output vector of the node of the interval operation layer in Formula (12); Q^{l} denotes a left permutation matrix, and the fuzzy subsequent parameters before and after the ascending arrangement of the left end points of the KM descent algorithm are transformed into each other through the permutation matrix Qₗ; Qᵣ denotes a right permutation matrix, and the fuzzy subsequent parameters before and after the ascending arrangement of the right end points of the KM descent algorithm are transformed into each other through the permutation matrix Qᵣ; E₁, E₂, E₃ and E₄ denote an upper interval unit matrix of a left point, a lower interval unit matrix of a left point, a lower interval unit matrix of a right point, and an upper interval unit matrix of a right point, respectively.

eᵢ¹∈ [symbol] ^{L},εᵢ²∈ [symbol] ^{M-L},eᵢ³∈ [symbol] ^{R},εᵢ⁴∈ [symbol] ^{M-R} denote an interval unit vector of a left point, an interval unit vector of a left point, an interval unit vector of a right point, and an interval unit vector of a right point. ri, si, rᵣ, sᵣ denote an interval unit vector of a left point, an interval unit vector of a left point, an interval unit vector of a right point, and an interval unit vector of a right point; L and R denote left and right turning points determined by the KM descent algorithm; [symbol] denotes a natural number; and [symbol] . denotes a positive integer.

Specifically, the sixth layer of the second structure of the six-layer interval type-II fuzzy neural network model is the output layer. The output corresponding to the unique node of the output layer is:

$$\begin{matrix}\hat{y} ( k ) = {\sum }_{j = 1}^{M} {ω}_{l}^{j} ( k ) [ {C}^{j} ( {z}^{- 1} ) θ ( k - 1 ) ] + {\sum }_{j = 1}^{M} {ω}_{r}^{j} ( k ) [ {C}^{j} ( {z}^{- 1} ) θ ( k - 1 ) ] + ξ ( k ) \\ {ω}_{l}^{j} ( k ) = \frac{{( {M}_{1}^{T} \overline{f} )}^{i} + {( {M}_{2}^{T} \underline{f} )}^{i}}{2 ( {\overline{f}}^{T} {R}_{l} + {\underline{f}}^{T} {S}_{l} )} , {ω}_{r}^{j} ( k ) = \frac{{( {M}_{3}^{T} \underline{f} )}^{i} + {( {M}_{4}^{T} \overline{f} )}^{i}}{2 ( {\underline{f}}^{T} {R}_{r} + {\overline{f}}^{T} {S}_{r} )} \\ {M}_{1} = {Q}_{l}^{T} {E}_{1}^{T} {E}_{1} {Q}_{l} , {M}_{2} = {Q}_{l}^{T} {E}_{2}^{T} {E}_{2} {Q}_{l} , {M}_{3} = {Q}_{r}^{T} {E}_{3}^{T} {E}_{3} {Q}_{r} , {M}_{4} = {Q}_{r}^{T} {E}_{4}^{T} {E}_{4} {Q}_{r} \\ {R}_{1} = {Q}_{l}^{T} {r}_{l} , {S}_{l} = {Q}_{l}^{T} {s}_{l} , {R}_{r} = {Q}_{r}^{T} {r}_{r} , {S}_{r} = {Q}_{r}^{T} , {s}_{r}\end{matrix} \qquad ( 15 )$$

where ŷ(k) denotes the predicted value of the NOₓ concentration at the SCR outlet at moment k; δ(k) denotes the noise variable at moment k; C^{j}(z⁻¹)θ(k−1) denotes the output vector of the fuzzy subsequent layer in Formula (13); ωᵢ^{j}(k) denotes the normalized value of the left interval membership of the j-th node of the fuzzy subsequent layer at moment k in Formula (13); ωᵣ^{j}(k) a c denotes the normalized value of the right interval membership of the j-th node of the fuzzy subsequent layer at moment k in Formula (13); ι̅ denotes the upper interval output vector of the node of the interval operation layer in Formula (12); ι denotes the lower interval output vector of the node of the interval operation layer in Formula (12); Qₗ denotes a left permutation matrix, and the fuzzy subsequent parameters before and after the ascending arrangement of the left end points of the KM descent algorithm are transformed into each other through the permutation matrix Qₗ; Qᵣ denotes a right permutation matrix, and the fuzzy subsequent parameters before and after the ascending arrangement of the right end points of the KM descent algorithm are transformed into each other through the permutation matrix Qᵣ; E₁, E₂, E₃ and E₄ denote an upper interval unit matrix of a left point, a lower interval unit matrix of a left point, a lower interval unit matrix of a right point, and an upper interval unit matrix of a right point, respectively; M₁, M₂, M₃ and M₄ denote an upper interval permutation matrix of a left point, a lower interval permutation matrix of a left point, a lower interval permutation matrix of a right point, and an upper interval permutation matrix of a right point, respectively.

Rₗ, Sₗ, Rᵣ, Sᵣ denote an upper interval permutation unit vector of a left point, a lower interval permutation unit vector of a left point, a lower interval permutation unit vector of a right point, and an upper interval permutation unit vector of a right point.

S13: The parameters of the type-II fuzzy layer of the second structure of the six-layer interval type-II fuzzy neural network model of the SCR reaction of diesel vehicle exhaust are calibrated using the gradient descent method.

Specifically, the error objective function of the second structure of the six-layer interval type-II fuzzy neural network model is selected as:

$${Θ}_{2} ( k ) = \frac{1}{2} {( y ( k ) - \hat{y} ( k ) )}^{2} = \frac{1}{2} {Δ}^{2} ( k ) \qquad ( 16 )$$

where Θ₂(k) denotes one half of the square of the error between the measured value of the NOₓ concentration at the SCR outlet and the predicted value of the NOₓ concentration at the SCR outlet at moment k; y(k) denotes the measured value of the NOₓ concentration at the SCR outlet at moment k; ŷ(k) denotes the predicted value of the NOₓ concentration at the SCR outlet at moment k; Δ(k)=y(k)−ŷ(k) denotes the predicted error of the NOₓ concentration at the SCR outlet at moment k.

Specifically, the parameter identification algorithm of the type-II fuzzy layer of the second structure of the six-layer interval type-II fuzzy neural network model is:

$$\begin{matrix}{m}_{i}^{j} ( k + 1 ) = {m}_{i}^{j} ( k ) - η \frac{\partial {Θ}_{2} ( k )}{\partial {m}_{i}^{j} ( k )} = {m}_{i}^{j} ( k ) + η ( y ( k ) - \hat{y} ( k ) ) \frac{\partial \hat{y} ( k )}{\partial {m}_{i}^{j} ( k )} \\ {σ}_{i}^{j} ( k + 1 ) = {σ}_{i}^{j} ( k ) - η \frac{\partial {Θ}_{2} ( k )}{\partial {σ}_{i}^{j} ( k )} = {σ}_{i}^{j} ( k ) + η ( y ( k ) - \hat{y} ( k ) ) \frac{\partial \hat{y} ( k )}{\partial {σ}_{i}^{j} ( k )} \\ {δ}_{i}^{j} ( k + 1 ) = {δ}_{i}^{j} ( k ) - η \frac{\partial {Θ}_{2} ( k )}{\partial {δ}_{i}^{j} ( k )} = {δ}_{i}^{j} ( k ) + η ( y ( k ) - \hat{y} ( k ) ) \frac{\partial \hat{y} ( k )}{\partial {δ}_{i}^{j} ( k )} \\ \frac{\partial \hat{y} ( k )}{\partial {m}_{i}^{j} ( k )} = \frac{{\overline{f}}^{j} ( {θ}_{i} - {m}_{i}^{j} ( k ) )}{2 {( {σ}_{i}^{j} ( k ) + {δ}_{i}^{j} ( k ) )}^{2}} ( \frac{{( {M}_{1} {ϑ}_{l} )}^{j} - {( {y}_{l} ( k ) {R}_{l} )}^{j}}{{\overline{f}}^{\overline{i}} {R}_{l} + {\underline{f}}^{\overline{i}} {S}_{l}} + \frac{{( {M}_{4} {ϑ}_{r} )}^{j} - {( {y}_{r} ( k ) {S}_{r} )}^{j}}{{\underline{f}}^{\overline{i}} {R}_{r} + {\overline{f}}^{\overline{i}} {S}_{r}} ) + \frac{{\underline{f}}^{j} ( {θ}_{i} - {m}_{i}^{j} ( k ) )}{2 {( {σ}_{i}^{j} ( k ) + {δ}_{i}^{j} ( k ) )}^{2}} ( \frac{{( {M}_{2} {ϑ}_{l} )}^{j} - {( {y}_{l} ( k ) {S}_{l} )}^{j}}{{\overline{f}}^{\overline{i}} {R}_{l} + {\underline{f}}^{\overline{i}} {S}_{l}} + \frac{{( {M}_{3} {ϑ}_{r} )}^{j} - {( {y}_{r} ( k ) {R}_{r} )}^{j}}{{\underline{f}}^{\overline{i}} {R}_{r} + {\overline{f}}^{\overline{i}} {S}_{r}} ) \\ \frac{\partial \hat{y} ( k )}{\partial {σ}_{i}^{j} ( k )} = \frac{{\overline{f}}^{j} ( {θ}_{i} - {m}_{i}^{j} ( k ) )}{2 {( {σ}_{i}^{j} ( k ) + {δ}_{i}^{j} ( k ) )}^{3}} ( \frac{{( {M}_{1} {ϑ}_{l} )}^{j} - {( {y}_{l} ( k ) {R}_{l} )}^{j}}{{\overline{f}}^{\overline{i}} {R}_{l} + {\underline{f}}^{\overline{i}} {S}_{l}} + \frac{{( {M}_{4} {ϑ}_{r} )}^{j} - {( {y}_{r} ( k ) {S}_{r} )}^{j}}{{\underline{f}}^{\overline{i}} {R}_{r} + {\overline{f}}^{\overline{i}} {S}_{r}} ) + \frac{{\underline{f}}^{j} ( {θ}_{i} - {m}_{i}^{j} ( k ) )}{2 {( {σ}_{i}^{j} ( k ) + {δ}_{i}^{j} ( k ) )}^{3}} ( \frac{{( {M}_{2} {ϑ}_{l} )}^{j} - {( {y}_{l} ( k ) {S}_{l} )}^{j}}{{\overline{f}}^{\overline{i}} {R}_{l} + {\underline{f}}^{\overline{i}} {S}_{l}} + \frac{{( {M}_{3} {ϑ}_{r} )}^{j} - {( {y}_{r} ( k ) {R}_{r} )}^{j}}{{\underline{f}}^{\overline{i}} {R}_{r} + {\overline{f}}^{\overline{i}} {S}_{r}} ) \\ \frac{\partial \hat{y} ( k )}{\partial {δ}_{i}^{j} ( k )} = \frac{{\overline{f}}^{j} ( {θ}_{i} - {m}_{i}^{j} ( k ) )}{2 {( {σ}_{i}^{j} ( k ) + {δ}_{i}^{j} ( k ) )}^{3}} ( \frac{{( {M}_{1} {ϑ}_{l} )}^{j} - {( {y}_{l} ( k ) {R}_{l} )}^{j}}{{\overline{f}}^{\overline{i}} {R}_{l} + {\underline{f}}^{\overline{i}} {S}_{l}} + \frac{{( {M}_{4} {ϑ}_{r} )}^{j} - {( {y}_{r} ( k ) {S}_{r} )}^{j}}{{\underline{f}}^{\overline{i}} {R}_{r} + {\overline{f}}^{\overline{i}} {S}_{r}} ) - \frac{{\underline{f}}^{j} ( {θ}_{i} - {m}_{i}^{j} ( k ) )}{2 {( {σ}_{i}^{j} ( k ) + {δ}_{i}^{j} ( k ) )}^{3}} ( \frac{{( {M}_{2} {ϑ}_{l} )}^{j} - {( {y}_{l} ( k ) {S}_{l} )}^{j}}{{\overline{f}}^{\overline{i}} {R}_{l} + {\underline{f}}^{\overline{i}} {S}_{l}} + \frac{{( {M}_{3} {ϑ}_{r} )}^{j} - {( {y}_{r} ( k ) {R}_{r} )}^{j}}{{\underline{f}}^{\overline{i}} {R}_{r} + {\overline{f}}^{\overline{i}} {S}_{r}} )\end{matrix} \qquad ( 17 )$$

where mᵢ^{j}(k+1) denotes the updated value of the center of the interval type-II Gaussian fuzzy membership function \tilde{F}ᵢ^{j} of the type-II fuzzy layer at moment k+1; mᵢ^{j}(k) denotes the un-updated value of the center of the interval type-II Gaussian fuzzy membership function \tilde{F}ᵢ^{j} of the type-II fuzzy layer at moment k; σᵢ^{j})k+1) denotes the updated value of the width of the interval type-II Gaussian fuzzy membership function \tilde{F}ᵢ^{j} of the type-I fuzzy layer at moment k+1; σᵢ^{j}(k) denotes the un-updated value of the width of the interval type-II Gaussian fuzzy membership function \tilde{F}ᵢ^{j} of the type-II fuzzy layer at moment k; δᵢ^{j}(k+1) denotes the updated value of the width interval of the interval type-II Gaussian fuzzy membership function \tilde{F}ᵢ^{j} of the type-II fuzzy layer at moment k+1; δᵢ^{j}(k): denotes the un-updated value of the width interval of the interval type-II Gaussian fuzzy membership function \tilde{F}ᵢ^{j} of the type-II fuzzy layer at moment k; ∂Θ₂(k)/∂mᵢ^{j}(k) denotes the derivative of the error objective function Θ₂(k) on mᵢ^{j}(k) in Formula (6); ∂Θ₂(k)/∂σᵢ^{j}(k) denotes the derivative of the error objective function Θ₂(k) on σᵢ^{j}(k)| in Formula (6); ∂Θ₂(k)/∂δᵢ^{j}(k) denotes the derivative of the error objective function Θ₂(k) on δᵢ^{j}(k) in Formula (6); η denotes the learning rate of the gradient descent method, in which the value is in the range of 0 to 1; ŷ(k) denotes the predicted value of the NOₓ concentration at the SCR outlet at moment k; ∂Θ₂(k)/∂mᵢ^{j}(k) denotes the derivative of the predicted value of the NOₓ concentration at the SCR outlet on mᵢ^{j}(k) at moment k; ∂Θ₂(k)/∂σᵢ^{j}(k) denotes the derivative of the predicted value of the NOₓ concentration at the SCR outlet on σᵢ^{j}(k) at moment k; ∂Θ₂(k)/∂δᵢ^{j}(k) denotes the derivative of the predicted value of the NOₓ concentration at the SCR outlet on δᵢ^{j}(k) at moment k. ι̅ⁱ denotes the upper membership interval operation value of the j-th node of the interval operation layer in Formula (12); ιⱼ denotes the upper membership interval operation value of the j-th node of the interval operation layer in Formula (12); ι̅ denotes the upper interval output vector of the node of the interval operation layer in Formula (12); ι denotes the lower interval output vector of the node of the interval operation layer in Formula (12); M₁, M₂, M₃ and M₄ denote an upper interval permutation matrix of a left point, a lower interval permutation matrix of a left point, a lower interval permutation matrix of a right point, and an upper interval permutation matrix of a right point, respectively.

Rₗ, Sₗ, Rᵣ, Sᵣ denote an upper interval permutation unit vector of a left point, a lower interval permutation unit vector of a left point, a lower interval permutation unit vector of a right point, and an upper interval permutation unit vector of a right point. yₗ(k) denotes the output of the node on the left side of the descent layer in Formula (14); yᵢ(k) the denotes output of the node on the right side of the descent layer in Formula (14).

S14: The parameters of the coefficient of the fuzzy subsequent layer of the second structure of the six-layer interval type-II fuzzy neural network model of SCR reaction of diesel vehicle exhaust are calibrated using the recursive least square method.

Specifically, the second structure of the six-layer interval type-II fuzzy neural network model of SCR reaction of diesel vehicle exhaust is rewritten in a vector form as:

$$\hat{y} ( k ) = {ξ}^{T} ( k - 1 ) \hat{β} ( k ) \qquad ( 18 )$$

$$where$$

$$\{ \begin{matrix}ξ ( k - 1 ) = {[ {ξ}_{1}^{T} ( k - 1 ) , L , {ξ}_{M}^{T} ( k - 1 ) ]}^{T} \\ \hat{β} ( k ) = {[ {β}_{1}^{T} ( k ) , L , {β}_{M}^{T} ( k ) ]}^{T} \\ {ξ}_{j} ( k - 1 ) = [ {ω}_{l}^{j} ( k ) y ( k - 1 ) , L , {ω}_{l}^{j} ( k ) y ( k - {n}_{y} ) , {ω}_{l}^{j} ( k ) {x}_{1} ( k - 1 ) , L , {ω}_{l}^{j} ( k ) {x}_{1} ( k - {n}_{{x}_{1}} ) , \\ {ω}_{l}^{j} ( k ) {x}_{2} ( k - 1 ) , L , {ω}_{l}^{j} ( k ) {x}_{2} ( k - {n}_{{x}_{2}} ) , {ω}_{l}^{j} ( k ) {x}_{3} ( k - 1 ) , L , {ω}_{l}^{j} ( k ) {x}_{3} ( k - {n}_{{x}_{3}} ) , \\ {ω}_{r}^{j} ( k ) y ( k - 1 ) , L , {ω}_{r}^{j} ( k ) y ( k - {n}_{y} ) , {ω}_{r}^{j} ( k ) {x}_{1} ( k - 1 ) , L , {ω}_{r}^{j} ( k ) {x}_{1} ( k - {n}_{{x}_{1}} ) , \\ {{ω}_{r}^{j} ( k ) {x}_{2} ( k - 1 ) , L , {ω}_{r}^{j} ( k ) {x}_{2} ( k - {n}_{{x}_{2}} ) , {ω}_{r}^{j} ( k ) {x}_{3} ( k - 1 ) , L , {ω}_{r}^{j} ( k ) {x}_{3} ( k - {n}_{{x}_{3}} ) ]}^{T} \\ {β}_{j} ( k ) = [ {a}_{1 , l}^{j} , L , {a}_{{n}_{y} , l}^{j} , {b}_{1 , l}^{j} , L , {b}_{{n}_{x 1} , l}^{j} , {d}_{11 , l}^{j} , L , {d}_{1 {n}_{x 2} , l}^{j} , {d}_{21 , l}^{j} , L , {d}_{2 {n}_{x 3} , l}^{j} , \\ {{a}_{1 , r}^{j} , L , {a}_{{n}_{y} , r}^{j} , {b}_{1 , r}^{j} , L , {b}_{{n}_{x 1} , r}^{j} , {d}_{11 , r}^{j} , L , {d}_{1 {n}_{x 2} , r}^{j} , {d}_{21 , r}^{j} , L , {d}_{2 {n}_{x 3} , r}^{j} ]}^{T}\end{matrix}$$

where ξ(k−1) denotes the known parameter vector at moment k−1; ξⱼ^{T}(k−1) denotes the j-th known parameter element at moment k−1, j=1, K, M; \hat{β}(k) denotes the coefficient vector of the fuzzy subsequent layer that needs to be updated at moment k; βⱼ(k); denotes the j-th coefficient element of the fuzzy subsequent layer that needs to be updated at moment k, j=1, K, M; ωᵢ^{j}(k) denotes the normalized value of the left interval membership of the j-th node of the fuzzy subsequent layer at moment kin Formula (13); and denotes the normalized value of the right interval membership of the j-th node of the fuzzy subsequent layer at moment k in Formula (13).

y(k−i) denotes the measured value of the NOₓ concentration at the SCR outlet at moment k−i. x₁(k−i) denotes the measured value of urea injection amount at moment k−i; x₂(k−i): denotes the measured value of the NOₓ concentration at the SCR inlet at moment k−i; x₃(k−i) denotes the measured value of the NH₃ escape concentration after SCR at moment k−i;

$$a \text{?} , a \text{?} , a \text{?}$$

$$\text{?} \text{indicates text missing or illegible when filed}$$

denote the first coefficient, the second coefficient and the n_{y}-th coefficient of the left interval polynomial Aₗ^{j}(z⁻¹) of the NOₓ concentration y at the SCR outlet, respectively;

$$a \text{?} , a \text{?} , a \text{?}$$

$$\text{?} \text{indicates text missing or illegible when filed}$$

denote the first coefficient, the second coefficient and the n_{y}-th coefficient of the right interval polynomial Aᵣ^{j}(z⁻¹) of the NOₓ concentration at the SCR outlet, respectively;

$$b \text{?} , b \text{?} , b \text{?}$$

$$\text{?} \text{indicates text missing or illegible when filed}$$

denote the first coefficient, the second coefficient and the nₓ_{₁}-th coefficient of the left interval polynomial Bₗ^{j}(z⁻¹) of the urea injection amount x₁, respectively;

$$b \text{?} , b \text{?} , b \text{?}$$

$$\text{?} \text{indicates text missing or illegible when filed}$$

denote the first coefficient, the second coefficient and the nₓ_{₁}-th coefficient of the right interval polynomial Bᵣ^{j}(z⁻¹) of the urea injection amount x₁, respectively;

$$d \text{?} , d \text{?} , d \text{?}$$

$$\text{?} \text{indicates text missing or illegible when filed}$$

denote the first coefficient, the second coefficient and the nₓ_{₂}-th coefficient of the left interval polynomial D_{1,j}^{j}(z⁻¹) of the NOₓ concentration x₂ at the SCR outlet, respectively;

$$d \text{?} , d \text{?} , d \text{?}$$

$$\text{?} \text{indicates text missing or illegible when filed}$$

denote the first coefficient, the second coefficient and the nₓ_{₂}-th coefficient of the right interval polynomial D_{1,r}^{j}(z⁻¹) of the NOₓ concentration x₂ at the SCR inlet, respectively;

$$d \text{?} , d \text{?} , d \text{?}$$

$$\text{?} \text{indicates text missing or illegible when filed}$$

denote the first coefficient, the second coefficient and the nₓ_{₃}-th coefficient of the left interval polynomial D_{2,l}^{j}(z⁻¹) of the NH₃ escape concentration x₃ at the SCR outlet, respectively;

$$d \text{?} , d \text{?} , d \text{?}$$

$$\text{?} \text{indicates text missing or illegible when filed}$$

denote the first coefficient, the second coefficient and the nₓ_{₃}-th coefficient of the right interval polynomial D_{2,r}^{j}(z⁻¹ of the NH₃ escape concentration x₃ at the SCR outlet, respectively.

The fuzzy subsequent layer coefficient vector \hat{β}(k) that needs to be updated is iteratively updated using the following recursive least square method:

$$\begin{matrix}\hat{β} ( k ) = \hat{B} ( k - 1 ) + K ( k ) [ y ( k ) - {ξ}^{T} ( k - 1 ) \hat{β} ( k - 1 ) ] \\ K ( k ) = \frac{P ( k - 1 ) ξ ( k - 1 )}{1 + {ξ}^{T} ( k - 1 ) P ( k - 1 ) ξ ( k - 1 )} \\ P ( k ) = P ( k - 1 ) - K ( k ) {ξ}^{T} ( k - 1 ) P ( k - 1 )\end{matrix} \qquad ( 19 )$$

where \hat{β}(k) denotes the coefficient vector of the fuzzy subsequent layer that needs to be updated at moment k; \hat{β}(k−1) denotes the coefficient vector of the fuzzy subsequent layer at moment k−1; K(k) denotes the known gain matrix at moment k; P(k−1) denotes the covariance matrix at moment k−1; P(k) denotes the covariance matrix updated at moment k.

Further, the steps of calibrating parameters of S13 and S14 are repeated until the error objective number in Formula (16) satisfies:

Θ₂(k)≤λ₂ (20)

where λ₂: denotes the prediction accuracy of the second structure of the six-layer interval type-II fuzzy neural network model.

Specifically, after the prediction accuracy of the second structure of the six-layer interval type-II fuzzy neural network model satisfies Formula (20), the parameters of the type-II fuzzy layer in the Formula (17) and the parameters of the fuzzy subsequent layer in the Formula (19) are fixed parameters of the second structure of the six-layer interval type-II fuzzy neural network model. In this way, the modeling of the SCR reaction process of diesel vehicle exhaust is completed.

The above is only the embodiment of the present disclosure, which does not limit the patent scope of the present disclosure. Any equivalent structure or equivalent process transformation made by using the contents of the specification and accompanying drawings of the present disclosure, which is directly or indirectly applied to other related technical fields, is equally included in the patent scope of protection of the present disclosure.

What is claimed is:

1. An intelligent modeling method of a Selective Catalytic Reduction (SCR) reaction process of diesel vehicle exhaust, comprising:
S1: acquiring a time sequence data set of an SCR reaction process of diesel vehicle exhaust for modeling, wherein the time sequence data set comprises an input variable sequence and an output variable sequence; and
S2: selecting a multi-layer type-II fuzzy neural network as an SCR reaction process model of diesel vehicle exhaust based on the input variable sequence and the output variable sequence obtained in S1;
wherein establishing the SCR reaction process model of diesel vehicle exhaust in the Step S2 comprises the following steps:
A1: selecting a type-II fuzzy membership function parameter and a fuzzy subsequent layer membership function, selecting a descent algorithm, and determining the number of nodes of a multi-layer structure;
A2: identifying antecedent parameters of a type-II fuzzy neural network model in an SCR reaction interval of diesel vehicle exhaust using a gradient descent method; and
A3: identifying antecedent parameters of the type-II fuzzy neural network model in the SCR reaction interval of diesel vehicle exhaust using a recursive least square method;
wherein the input variable sequence in the Step S1 comprises one or more of an NOₓ concentration at an SCR inlet, a urea injection amount, an NH₃ escape concentration after SCR and an SCR catalyst temperature;
the output variable sequence in the Step S1 comprises an NOₓ concentration at an SCR outlet; and
the multi-layer type-II fuzzy neural network model in the Step S2 comprises an input layer, a type-II fuzzy layer, an interval operation layer, a fuzzy subsequent layer, a descent layer and an output layer.

2. The intelligent modeling method of the SCR reaction process of diesel vehicle exhaust according to claim 1, wherein the time sequence of the input variable sequence and the output variable sequence in the Step S1 comprise a sequence consisting of a measured value at a current moment and a measured value at a historical moment; and
a method of acquiring the time sequence data in the Step S1 comprises one of an engine bench test method and a vehicle field test method of a portable emission measurement system.

3. The intelligent modeling method of the SCR reaction process of diesel vehicle exhaust according to claim 1, wherein the number of nodes in the input layer is one or two or more of time sequence data of an NOₓ concentration at an SCR inlet, a urea injection amount, an NH₃ escape concentration after SCR and an SCR catalyst temperature; and
the number of nodes of a type-II fuzzy layer, an interval operation layer and a fuzzy subsequent layer of the multi-layer type-II fuzzy neural network model in the Step S2 is the same, which is specified by artificial experience or obtained by parameter optimization.

4. The intelligent modeling method of the SCR reaction process of diesel vehicle exhaust according to claim 3, wherein the number of nodes of the descent layer of the multi-layer type-II fuzzy neural network model in the Step S2 is determined according to the descent algorithm;
the descent algorithm comprises a first descent algorithm and a second descent algorithm;
the first descent algorithm determines that the number of nodes of the descent layer of the multi-layer type-II fuzzy neural network model is 2; and
the second descent algorithm determines that the number of nodes of the descent layer of the multi-layer type-II fuzzy neural network model is the same as that the number of nodes of the fuzzy subsequent layer.

5. The intelligent modeling method of the SCR reaction process of diesel vehicle exhaust according to claim 4, wherein the first descent algorithm is an iterative algorithm, comprising any one of a Karnik-Mendel algorithm, an enhanced Karnik-Mendel algorithm, an enhanced Karnik-Mendel algorithm with new initialization algorithm, an iterative algorithm with stop condition algorithm, an enhanced iterative algorithm with stop condition algorithm, and an enhanced opposite direction searching algorithm; and
the second descent algorithm is a closed-loop algorithm, comprising any one of a Wu-Tan algorithm, a Nie-Tan algorithm, a Du-Ying algorithm and a Begian-Melek-Mendel algorithm.

6. The intelligent modeling method of the SCR reaction process of diesel vehicle exhaust according to claim 3, wherein the type-II fuzzy membership function in the Step A1 comprises one of an interval type-II Gaussian membership function, a generalized type-II Gaussian membership function, an interval type-II triangular membership function, a generalized type-II triangular membership function, an interval type-II trapezoidal membership function, a generalized type-II trapezoidal membership function, an interval type-II bell-shaped membership function and a generalized type-II bell-shaped membership function.

7. The intelligent modeling method of the SCR reaction process of diesel vehicle exhaust according to claim 1, wherein the fuzzy subsequent layer membership function of the type-II fuzzy neural network comprises any one of a single-valued polynomial, an interval type-II fuzzy polynomial and a generalized type-II fuzzy polynomial.

8. The intelligent modeling method of the SCR reaction process of diesel vehicle exhaust according to claim 1, wherein establishing the type-II fuzzy neural network model needs to calibrate the type-II fuzzy membership function parameter of the type-II fuzzy layer and the coefficients of the fuzzy subsequent layer polynomial.

9. The intelligent modeling method of the SCR reaction process of diesel vehicle exhaust according to claim 1, wherein A3 further comprises using the time sequence data set of the SCR reaction process of diesel vehicle exhaust to train the type-II fuzzy neural network model antecedent parameter obtained in A2 and the type-II fuzzy neural network model subsequent parameter obtained in A3; and determining the parameters of the type-II fuzzy layer and the fuzzy subsequent layer of the type-II fuzzy neural network model calibrated to verify an minimum error and the descent algorithm as the multi-layer type-II fuzzy neural network model of the SCR reaction process of diesel vehicle exhaust.

10. The intelligent modeling method of the SCR reaction process of diesel vehicle exhaust according to claim 9, wherein a training algorithm comprises one of a gradient descent method, a Newton method, a quasi-Newton method, a steepest descent method, a simulated annealing method, a genetic algorithm, an ant colony algorithm, a particle swarm algorithm, a least square method and a recursive least square method.
